(19) United States
(12) Patent Application Publication
(10) Pub. No.: US 2023/0399485 A1
(43) Pub. Date: Dec. 14, 2023

(54) IMPROVED METHOD FOR RECYCLING PET BY ALCOHOLYSIS

(71) Applicant: RECYC'ELIT, Chasse-Sur-Rhône (FR)

(72) Inventor: Raouf Medimagh, Lyon (FR)

(21) Appl. No.: 18/253,439

(22) PCT Filed: Nov. 24, 2021
(86) PCT No.: PCT/FR2021/052085

§ 371 Date: May 18, 2023

(30) Foreign Application Priority Data
Nov. 24, 2020 (FR) 2012085

(51) Int. Cl. C08J 11/22; C08J 11/28

(52) U.S. Cl. CPC C08J 11/22; C08J 11/28; C08J 2367/02

(57) ABSTRACT

The present invention relates to the field of recycling PET-type plastics commonly used for the manufacture of disposable plastic bottles, food trays, textiles, etc. More specifically, it relates to a method for converting PET into dimethyl terephthalate (DMT) in a few hours (less than 5 hours) by means of a complete reaction resulting in a product free of impurities. The depolymerisation step is carried out in the presence of a monoalcohol as well as an organic base with a guanidine or amidine unit, and a second base which may be either an inorganic base or an ether oxide. These two bases are present in catalytic amounts relative to the amount of PET to be treated.

### CROSS-REFERENCE TO RELATED APPLICATIONS

This application is a national phase entry under 35 U.S.C. § 371 of International Patent Application PCT/FR2021/052085, filed Nov. 24, 2021, designating the United States of America and published as International Patent Publication WO 2022/112715 A1 on Jun. 2, 2022, which claims the benefit under Article 8 of the Patent Cooperation Treaty to French Patent Application Serial No. 2012085, filed Nov. 24, 2020.

### TECHNICAL FIELD

The present disclosure relates to the field of recycling PET-type plastics commonly used for the manufacture of disposable plastic bottles, food trays, textiles, etc. More specifically, it relates to a method for converting PET into dimethyl terephthalate (DMT) in a few hours (less than 5 hours) by means of a complete reaction resulting in a product free of impurities. The depolymerization step is carried out in the presence of a monoalcohol as well as an organic base with a guanidine or amidine unit, and a second base, which may be either an inorganic base or an ether oxide. These two bases are present in catalytic quantities relative to the amount of PET to be treated.

### BACKGROUND

PET recycling is a major environmental matter and thus represents a commercial opportunity due to its generalized use, its abundance and its durability. However, the recycling of plastics is complex and varies according to the type of polymer, the design of the packaging and the type of product.

The main impediment for the use of recycled plastic materials is the contamination of waste streams with different types of polymers that are not compatible with one another. As a result, it is often not possible to add recycled PET-type plastic to virgin polymer without reducing certain quality attributes, such as color, clarity or impact resistance. For this reason, the ability to replace a virgin polymer with recycled PET depends greatly on the purity of the recycled product and the requirements of the final product.

According to the principle of chemical recycling, PET can be depolymerized by methanolysis or glycolysis, and the monomers thus obtained can be reused to generate new PET polymers referred to as “recycled PET.”

Depending on industrial needs, certain technologies for manufacturing PET resin resort to the use of terephthalic acid dimethyl ester (DMT).

Conventional methanolysis techniques use methods that are very energy intensive and costly in equipment. These methods use a supercritical phase at temperatures higher than 300° C. and at pressures between 5 and 10 bar of pressure, which induces structural changes in the molecular units of PET, in particular, isomerization or deterioration. These modified molecules may be toxic or generate disruptions during the production of recycled PET. The molecules are detrimental to the quality of the depolymerized product for these future applications.

Document WO2020/128218 describes a method for depolymerization of PET by alcoholysis using a monoalcohol such as methanol or ethanol and a base selected from sodium methoxide, KOH or NaOH in a stoichiometric amount relative to the PET.

It is known that the use of a base in catalytic amount relative to the mass of PET makes it possible to obtain DMT, but the kinetics of the reaction is quite slow. The reaction time is greater than 10 hours 30 minutes, during which time the reaction solution is heated continuously. By way of example, mention may be made of US2019/0256450 and WO2020/188359, which describe the depolymerization of PET into DMT in the presence of methanol and an alkoxide such as sodium methoxide. These methanolysis reactions take place at temperatures of between 25° C. and 100° C. These methods necessarily comprise a first stage of swelling the PET with chlorinated solvents or polar solvents such as DMSO or DMF or methanol. US2019/0256450 proposes reacting the PET with a base, sodium methoxide in catalytic amount, and methanol. The method described in WO2020/188359 is characterized by the sequential addition of methanol and methylate solutions several times after addition of sodium methoxide. The authors describe high PET production yields. US2019/390035 describes another approach for depolymerization by adding glycolate salt. The preparation of this salt comprises isolation and drying steps, which extend over one week.

For a person skilled in the art, the implementations of the methods described above obviously exhibit problems of industrial operability and feasibility regarding the safety aspect of an ATEX environment such as that of methanol under reflux, which requires complex precautions and expensive devices when introducing flammable products into the method.

Superbases with guanidine or amidine units described in the literature such as 1,8-diazabicyclo[5.4.0]undec-7-ene (DBU) and triazabicyclodecene (TBD) are considered to be effective organocatalysts and, in particular, in depolymerization reactions by glycolysis. Such a reaction is, for example, described by Hedrik et al. (WO2012121985). This is a glycolysis reaction conducted at 190° C., which does not lead to DMT but to bis(2-Hydroxyethyl) terephthalate (or BHET).

Horn et al. (2012) describes a reaction for depolymerization of PET by glycolysis, organocatalyzed by TBD or DBU at 160° C. The reaction is complete in 20 min with DBU and in 110 min with TBD. The reaction produces bis(2-Hydroxyethyl)terephthalate (BHET). However, this reaction does not work when a short-chain monoalcohol is used.

On the other hand, none of these patents reports the use of such bases with a guanidine or amidine unit to obtain DMT. Indeed, the use of methanol as solvolysis solvent does not give results in these methods. Generally, these are diols that are considered effective according to the prior art, since they act as co-catalysts in the solvolysis mechanism.

None of these methods is satisfactory, thus it is desirable to have improved PET recycling methods, at low cost and easily operable industrially, in order to facilitate the scaling-up of this recycling and broaden the fields of use for recycled PET.

### BRIEF SUMMARY

A new method has been developed for depolymerization by alcoholysis under mild conditions for recycling the polyethylene terephthalate (PET) polymer into terephthalate and monoethylene glycol ester (MEG) monomers. It is fast and gives access to a product in solid form that is directly reusable due to its purity, in particular, DMT in crystalline form.

This method for recycling plastic waste (PET) into a powder of terephthalate ester monomers comprises three steps:
a. a step of milling the waste to produce fragments,
b. a step of pre-treating the fragments to facilitate their depolymerization, and
c. a step of depolymerization of the PET in terephthalate ester and monoethylene glycol (MEG), in the presence of (i) an organic base comprising a guanidine or amidine unit such as DBU, TBD or the non-cyclic form such as tetramethylguanidine (TTMG) and (ii) an etheroxide base of the sodium methoxide or potassium methoxide type, or inorganic, of the sodium hydroxide or potassium hydroxide type,
and is characterized in that step c. is done:
   in the presence of an excess of monoalcohol relative to the amount of PET;
   in that the bases are present in catalytic amount relative to the amount of PET; and
   by heating between 25° C. and 80° C. for a period of between 30 minutes and 5 hours.

The method according to the present disclosure proposes to combine an etheroxide base of the sodium or potassium methoxide type or an inorganic base of the sodium or potassium hydroxide type with an organic base, both in catalytic amount relative to PET and a monoalcohol such as methanol, ethanol, propanol or butanol, and they are reacted under mild conditions. It has several advantages with regard to the methods described above, which are explained hereunder:

Remarkably, the depolymerization reaction is complete, fast and produces a high-purity terephthalate ester. This is particularly advantageous when the PET is depolymerized in DMT since the latter is then easily recyclable.

Indeed, the method is fast since the reaction is complete in less than 5 hours, and even in less than 2 hours under optimized conditions.

The depolymerization reaction is simple. Depolymerization and purification can be carried out in one and the same step. After completion of the reaction, the product obtained is directly a terephthalate ester (such as DMT) in the form of crystals, without being mixed with intermediate or degradation products that would be necessary to separate from the product of interest.

The yield of the method is high; at least 85%, in particular, for depolymerization of the PET into DMT.

In the particular case of the depolymerization of PET into DMT using methanol, the product obtained is 99.9% pure at the end of the reaction (after filtration and washing). There is therefore no need for subsequent purification. The DMT can be used directly after washing with methanol. Given its level of purity, it may be used in numerous applications, for remaking PET or any other type of technical resin involving this monomer. The choice of reagents and the fact that the reaction conditions are mild means that no isomerization reaction occurs, and that no degradation products that are detrimental to the quality of the product obtained are formed. When present, these secondary molecules to the reaction disrupt the polymerization reaction and a purification of the crude DMT is therefore necessary before it can be used.

This method is more economical and more environmentally friendly than the existing methods due to the fact that the bases are used in catalytic quantities relative to the amount of PET to be recycled, and that the reaction temperatures are less than 100° C., generally included between room temperature and 80° C. Furthermore, the pre-treatment step can be carried out in the presence of an aprotic solvent, as an alternative to solvents that are controversial with regard to environmental standards, without this affecting the effectiveness of the reaction, in particular, the reaction time.

From an ecological point of view, it should be noted that the depolymerization bath containing the solvent can be reused for a new treatment cycle once the product has been filtered. The bath can be used at least twice without affecting the effectiveness of the reaction.

### DETAILED DESCRIPTION

The present disclosure relates to a method for recycling waste from polyethylene terephthalate (PET) plastics into a powder of terephthalate ester monomers comprising three steps:
a. a step of milling the waste to produce fragments,
b. a step of pre-treating the fragments to facilitate their depolymerization,
c. a step of depolymerizing the PET in an ester of terephthalate and monoethylene glycol (MEG), in the presence of (i) an organic base comprising an amidine or guanidine unit and (ii) an etheroxide base of the sodium methoxide or potassium methoxide type, or inorganic base of the sodium hydroxide or potassium hydroxide type,
wherein step c. is done:
   in the presence of excess monoalcohol relative to the amount of PET;
   in that the bases are present in catalytic amount relative to the amount of PET; and
   by heating between 25° C. and 80° C. for a period of between 30 minutes and 5 hours.

“Catalytic amount” within the meaning of the present disclosure means a non-stoichiometric amount, which is to say, in a molar ratio of 1% to 49% relative to the amount of PET to be treated. The term “catalytic” also applies to a reagent that is found in its initial form at the end of the reaction (catalyst).

In a preferred embodiment, the catalytic amount of each of the bases is an amount of less than 30%, 25% and 20% relative to the amount of PET. More preferably, it is less than 15%, or even 10%. Very preferably, it is less than 5%, in particular, between 1 and 3%, for example, 1.5%.

The monoalcohol can be chosen, for example, from methanol, ethanol, propanol and butanol. The terephthalate ester obtained will depend on the monoalcohol used.

When the monoalcohol is methanol, dimethyl terephthalate (DMT) is obtained.

When the monoalcohol is ethanol, diethyl terephthalate (DET) is obtained.

When the monoalcohol is propanol, dipropyl terephthalate (DPT) is obtained.

When the monoalcohol is butanol, dibuthyl terephthalate (DBT) is obtained.

These different products find their application in the oil industry.

In a preferred embodiment of the present disclosure, the method makes it possible to obtain DMT. The reaction product is a DMT powder with a high purity on the order of 99%, in the form of crystals, which can be filtered and washed at the end of the polymerization step. Thus, this method is characterized by the fact that depolymerization and purification are carried out in a single step. The DMT monomers are recovered simply by filtering the solid present in the solvent bath, followed by washing with methanol.

The depolymerization step is preceded by a pre-treatment in order to facilitate the depolymerization reaction, and the access of the bases to the polymers. This pretreatment can be carried out in different ways, described in the prior art and well known to a person skilled in the art.

The pretreatment step may involve soaking without dissolving in a container ensuring continuous stirring of the PET fragments in a solvent solution. Different types of solvent may be used, alone or as a mixture, selected from:
aprotic polar solvents such as DMAc (dimethyl acetamide), dMf (dimethyl formamide), dimethyl sulfoxide (DMSO), 2-butanone or MEK (Methyl Ethyl Ketone), phenolic esters;
non-polar solvents such as biphenyl ethers or chlorinated agents such as dichloromethane, dichloroethane, tetrachloroethane or chlorobenzene;
cyclic or linear ethers such as dioxane, ethylene glycol, propylene glycol, etc.

In a preferred embodiment of the present disclosure, the solvent is a non-chlorinated aprotic solvent chosen from DMAc, DMF, 2-butanone (or MEK) and is used at a temperature of less than 50° and for a period of less than 18 hours. The soaking step is followed by a step of settling and by dewatering.

The pre-treatment step is essential for the depolymerization reaction to take place correctly so that it gives the expected results under the reaction conditions described below.

It is possible to carry out a step of alcohol-washing and drying the pretreated PET pieces before the depolymerization step.

The depolymerization step is done in the presence of two bases. The first is an organic base comprising an amidine unit, such as 1,8-Diazabicyclo[5.4.0]undec-7-ene (DBU), 1,5-Diazabicyclo(4.3.0)non-5-ene (DBN) or with a guanidine unit such as triazabicyclodecene (TBD), 1,1,3,3-Tetramethylguanidine (TTMG), guanidine hydrochloride. The second is an etheroxide base of the sodium methoxide type, or inorganic, of sodium hydroxide or potassium hydroxide type. Indeed, this set of bases has the advantage of being able to contribute to renewing the formation of the depolymerization catalyst in order to ensure the solvolysis in a rapid manner. As mentioned above, the use of the organic base alone in the presence of a monoalcohol does not allow the reaction to progress. In the same way, the use of the methoxide base alone in catalytic amounts does not allow the reaction to finish within the reaction times cited above for this method. The combination of this set of bases is therefore indispensable and has been reported nowhere else.

The unit of the organic base is represented by formula (I) below:

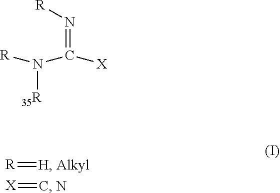

In a particular embodiment, the amount of base DBU is less than 2% relative to the molar amount of PET.

In another particular embodiment, the organic base is DBU and the ether oxide base is sodium methoxide in an amount of less than 5% relative to the molar amount of PET.

In another particular embodiment, the organic base is DBU and the inorganic base is potassium hydroxide; preferably both bases are used in an amount of less than 5% relative to the molar amount of PET.

In addition to the bases, the depolymerization reaction is done in the presence of a monoalcohol as a solvent. The monoalcohol is present in excess relative to the amount of PET. Thus, the amount of monoalcohol is at least 4 times (or even 5 times) higher in weight ratio to the amount of PET, typically between 4 times and 20 times, or even between 5 and 20 times. When the monoalcohol is methanol, the final product is therefore DMT. When the monoalcohol is ethanol, propanol or butanol, the final products are, respectively, DET, DPT and DBT.

The depolymerization reaction is carried out by heating between 25° C. and 80° C., preferably between 50° C. and 75° C., quite preferably between 60° C. and 70° C. The reaction time will depend on the temperature and the relative amount of the reagents involved relative to the amount of PET. The person skilled in the art knows how to adapt these parameters. Thus, the reaction time will generally be between 30 minutes and 5 hours and preferably between 1 hour 30 minutes and 3 hours, even more preferably 2 to 3 hours.

In a preferred embodiment, the reaction time will be 2 hours to 3 hours and the temperature of 55° C. to 70° C.

The method according to the present disclosure takes place at atmospheric pressure.

In some embodiments of the present disclosure, the method will be carried out under the following conditions:
in the presence of sodium methoxide in a molar ratio of 5% relative to the PET, of DBU in a molar ratio of 1.5% relative to the PET, and of methanol at a weight ratio of 5 times by heating for 4 hours at 70° C.;
in the presence of sodium methoxide and DBU, the two bases being in a molar ratio of 15% relative to the PET, and of methanol at a weight ratio of 10 times by heating for 3 hours at 70° C.;
in the presence of potassium hydroxide in a molar ratio of 15% relative to the PET, of DBU also in a molar ratio of 15% relative to the PET, and of methanol at a weight ratio of 20 times by heating for 3 hours at 70° C.;
in the presence of sodium methoxide in a molar ratio of 15% relative to the PET, of DBU in a molar ratio of 1.5% relative to the PET, and of methanol at a weight ratio of 10 times by heating for 3 hours at 70° C.;
in the presence of sodium methoxide in a molar ratio of 15% relative to the PET, of TBD in a molar ratio of 15% relative to the PET, and of methanol at a weight ratio of 20 times by heating for 2 hours 30 minutes at 70° C.

The yields obtained under these conditions are at least 80%.

At the end of the depolymerization step noted by the disappearance of the pieces of PET initially introduced, the DMT can be directly recovered by filtering and washing the cake obtained. It is 99% pure and can be used directly to generate PET again through reaction with ethylene glycol. The quality of the DMT regenerated by this method, then of the recycled PET obtained from this DMT allows for use in applications where high quality is required, for example, in a mixture with virgin PET or other polymers when the presence of contaminants would be harmful to the quality criteria, such as color, clarity or impact strength.

In the case where the pieces of PET introduced contain impurities constituted by metal, wood debris, plastics of different natures (polypropylene, polyethylene, PVC, etc.), it is possible to use a sieve whose porosity is at an intermediate size allowing the crystal powder (in particular, DMT) to pass through but preventing the same for the impurities mentioned above, which will not have reacted, given the selectivity of the method with respect to PET only. As a non-limiting example, a sieve with a porosity ranging from 0.5 to 1 mm could allow this operation.

### EXAMPLES

### Example 1: Recycling of the PET in DMT in the Presence of Sodium Methoxide and DBU in Catalytic Amounts, and Methanol

A quantity (5 g) of pieces of polyethylene terephthalate PET originating from food trays, after having been washed with water, is placed in a container containing dimethylacetamide DMAc (20 mL) in such a way that all the pieces of plastic are submerged. They are stirred for 2 hours 30 minutes. Next, the treated PET pieces are drained, optionally washed with alcohol and dried before being transferred to a 100 mL glass reactor. 25 mL of anhydrous methanol are added to the pretreated pieces followed by 0.9 mL of a sodium methoxide solution (25% in methanol) corresponding to a molar ratio of 15% sodium methoxide relative to the PET introduced. This operation is followed by adding 0.58 mL of DBU corresponding to a molar ratio of 15% relative to the PET introduced. After 180 minutes of reaction at 70° C., all the pieces of PET have disappeared leaving a white solid in solution. The crude reaction mixture is filtered through a filter paper or Buchner paper; the recovered liquid contains residual methanol and the monoethylene glycol produced from the depolymerization reaction and also the bases initially reacted. The white solid (DMT) that is recovered (3.6 g) is washed with methanol.

### Example 2: Recycling of the PET in DMT in the Presence of Potassium Hydroxide and DBU in Catalytic Amounts, and Methanol

A quantity (5 g) of pieces of polyethylene terephthalate PET originating from food trays, after having been washed with water, is placed in a container containing dimethylacetamide DMAc (20 mL) in such a way that all the pieces of plastics are submerged. They are stirred for 2 hours 30 minutes. Next, the treated PET pieces are drained, optionally washed with alcohol and dried before being transferred to a 100 mL glass reactor. 25 mL of anhydrous methanol are added to the pretreated pieces followed by 0.220 g of potassium hydroxide (KOH) corresponding to a molar ratio of 15% and a weight ratio of 5% relative to the PET introduced. This operation is followed by adding 0.58 mL of DBU corresponding to a molar ratio of 15% relative to the PET introduced. After 180 minutes, all the pieces of PET have disappeared, leaving a white solid in solution. The crude reaction mixture is filtered through a filter paper or Buchner paper; the recovered liquid contains residual methanol and the monoethylene glycol produced from the depolymerization reaction and also the bases initially reacted. The white solid (DMT) that is recovered (4.2 g) is washed with methanol (85% efficiency).

### Example 3: Recycling of the PET in DMT in the Presence of Sodium Methoxide and DBU in Catalytic Amounts, and Methanol

A quantity (1.25 g) of pieces of polyethylene terephthalate PET originating from food trays, after having been washed with water, is placed in a container containing dimethylacetamide DMAc in such a way that all the pieces of plastics are submerged. They are stirred for 2 hours 30 minutes. Next, the treated PET pieces are drained, optionally washed with alcohol and dried before being transferred to a 50 mL glass reactor. 15 mL of anhydrous methanol are added to the pretreated pieces followed by 0.44 mL of sodium methoxide (25% in methanol) corresponding to a molar ratio of 30% relative to the PET introduced. This operation is followed by the addition of 0.015 mL of DBU corresponding to a molar ratio of 1.5% relative to the PET introduced. After 180 minutes, all the pieces of PET have disappeared, leaving a white solid in solution. The crude reaction mixture is filtered through a filter paper or Buchner paper; the recovered liquid contains residual methanol and the monoethylene glycol produced from the depolymerization reaction and also the bases initially reacted. The white solid (DMT) that is recovered (1 g) is washed with methanol (84% efficiency).

### Example 4: Recycling of the PET in DMT in the Presence of Sodium Methoxide and TBD in Catalytic Amounts, and Methanol

A quantity (5 g) of pieces of polyethylene terephthalate PET originating from food trays, after having been washed with water, is placed in a container containing dimethylacetamide (DMAc) in such a way that all the pieces of plastic are submerged. They are stirred for 2 hours 30 minutes. Next, the treated PET pieces are drained, optionally washed with alcohol and dried before being transferred to a 150 mL glass reactor. 90 mL of anhydrous methanol are added to the pretreated pieces followed by 0.9 mL of sodium methoxide (25% in methanol) corresponding to a molar ratio of 30% relative to the PET introduced. This operation is followed by adding 0.54 mL of TBD corresponding to a molar ratio of 15% relative to the PET introduced. After 150 minutes, all the pieces of PET have disappeared, leaving a white solid in solution. The crude reaction mixture is filtered through a filter paper or Buchner paper; the recovered liquid contains residual methanol and the monoethylene glycol produced from the depolymerization reaction and also the bases initially reacted. The white solid (DMT) that is recovered (4 g) is washed with methanol (81% efficiency).

### Example 5: Recycling of the PET in DMT in the Presence of Sodium Methoxide and DBN in Catalytic Amounts, and Methanol

A quantity (5 g) of pieces of polyethylene terephthalate PET originating from food trays, after having been washed with water, is placed in a container containing dimethylacetamide (DMAc) in such a way that all the pieces of plastic are submerged. They are stirred for 2 hours 30 minutes. Next, the treated PET pieces are drained, optionally washed with alcohol and dried before being transferred to a 150 mL glass reactor. 90 mL of anhydrous methanol are added to the pretreated pieces followed by 0.9 mL of sodium methoxide (25% in methanol) corresponding to a molar ratio of 30% relative to the PET introduced. This operation is followed by the addition of 65 mg of TBD corresponding to a molar ratio of 2% relative to the PET introduced. After 150 minutes, all the pieces of PET have disappeared, leaving a white solid in solution. The crude reaction mixture is filtered through a filter paper or Buchner paper; the recovered liquid contains residual methanol and the monoethylene glycol produced from the depolymerization reaction and also the bases initially reacted. The white solid (DMT) that is recovered (4 g) is washed with methanol (81% efficiency).

The DMT obtained was repolymerized according to the conventional PET synthesis routes and made it possible to obtain very satisfactory technical specifications compared to a commercial DMT.

1. A method for recycling polyethylene terephthalate (PET) plastic waste into a powder of terephthalate ester monomers comprising three steps:
a. a step of milling the waste to produce fragments;
b. a step of pre-treating the fragments to facilitate their depolymerization; and
c. a step of depolymerizing the PET in an ester of terephthalate and monoethylene glycol (MEG), in the presence of (i) an organic base comprising an amidine or guanidine unit and (ii) an etheroxide base of the sodium methoxide or potassium methoxide type, or inorganic base of the sodium hydroxide or potassium hydroxide type;
wherein step c. is done:
in that the bases are present in catalytic amount relative to the amount of PET;
in the presence of excess monoalcohol relative to the amount of PET, and
by heating between 25° C. and 80° C. for a period of between 30 minutes and 5 hours.

2. The method of claim 1, wherein the pretreatment step is carried out in the presence of an aprotic solvent chosen from dimethyl acetamide (DMAc), dimethyl formamide (DMF) or Methyl Ketone (MEK).

3. The method of claim 1, wherein the organic base is of formula (I):

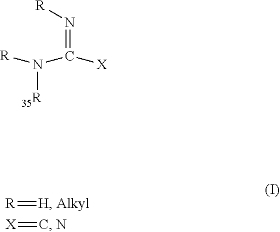

4. The method of claim 3, wherein the organic base is selected from (i) a base comprising an amidine unit selected from 1,8-Diazabicyclo[5.4.0]undec-7-ene (DBU), 1,5-Diazabicyclo(4.3.0)non-5-ene (DBN) or (ii) an organic base comprising a guanidine unit selected from triazabicyclodecene (TBD), 1,1,3,3-Tetramethylguanidine (TTMG), or guanidine hydrochloride.

5. The method of claim 1, wherein the inorganic base is selected from sodium or potassium methoxide, sodium hydroxide or potassium hydroxide.

6. The method of claim 1, wherein the monoalcohol is selected from methanol, ethanol, propanol or butanol.

7. The method of claim 6, wherein the monoalcohol is methanol and the terephthalate ester obtained is dimethyl terephthalate.

8. The method of claim 6, wherein the monoalcohol is ethanol and the terephthalate ester obtained is diethyl terephthalate.

9. The method of claim 6, wherein the monoalcohol is propanol and the obtained terephthalate ester is dipropyl terephthalate.

10. The method of claim 6, wherein the monoalcohol is butanol and the terephthalate ester obtained is dibuthyl terephthalate.

11. The method of claim 1, wherein the amount of the bases is less than 5%.

12. The method of claim 1, wherein the amount of methanol is at least 5 times greater in molar ratio than the amount of PET.

13. The method of claim 1, wherein the depolymerization step is carried out by heating between 50° C. and 70° C. for 1 hour 30 minutes to 3 hours.

14. The method of claim 1, further comprising a step of washing with alcohol and drying the pretreated PET pieces before the depolymerization step.

15. The method of claim 1, further comprising recovering of the DMT by filtration of the reaction medium and washing of a cake obtained.

16. A method for recycling polyethylene terephthalate (PET) plastic waste into a powder of terephthalate ester monomers, comprising:
milling the waste to produce fragments;
pre-treating the fragments to facilitate their depolymerization; and
depolymerizing the PET in an ester of terephthalate and monoethylene glycol (MEG), in the presence of (i) an organic base comprising an amidine or guanidine unit and (ii) an etheroxide base of at least one of sodium methoxide, potassium methoxide, sodium hydroxide, or potassium hydroxide;
wherein, during the depolymerization, the organic base and the etheroxide base are present in catalytic amount relative to the amount of PET, the depolymerization is carried out in the presence of excess monoalcohol relative to the amount of PET, and the PET is heated to a temperature between 25° C. and 80° C. for a period of between 30 minutes and 5 hours during the depolymerization.

17. The method of claim 16, wherein the pretreatment step is carried out in the presence of an aprotic solvent chosen from dimethyl acetamide (DMAc), dimethyl formamide (DMF) or Methyl Ketone (MEK).

18. The method of claim 16, wherein the organic base is of formula (I):

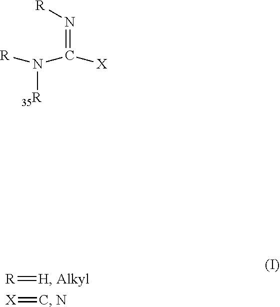

19. The method of claim 18, wherein the organic base is selected from (i) a base comprising an amidine unit selected from 1,8-Diazabicyclo[5.4.0]undec-7-ene (DBU), 1,5-Diazabicyclo(4.3.0)non-5-ene (DBN) or (ii) an organic base comprising a guanidine unit selected from triazabicyclodecene (TBD), 1,1,3,3-Tetramethylguanidine (TTMG), or guanidine hydrochloride.

20. The method of claim 16, wherein the inorganic base is selected from sodium or potassium methoxide, sodium hydroxide or potassium hydroxide.
